(19) United States
(12) Patent Application Publication
(10) Pub. No.: US 2022/0346950 A1
(43) Pub. Date: Nov. 3, 2022

(54) SYSTEMS AND METHODS FOR FLUID CONTROL

(71) Applicant: Edwards Lifesciences Corporation, Irvine, CA (US)

(72) Inventors: Tung T. Le, Costa Mesa, CA (US); Sonny Tran, Westminster, CA (US)

(21) Appl. No.: 17/812,669

(22) Filed: Jul. 14, 2022

Related U.S. Application Data
(63) Continuation of application No. PCT/US2021/013092, filed on Jan. 12, 2021.
(60) Provisional application No. 62/961,845, filed on Jan. 16, 2020.

(51) Int. Cl. A61F 2/24; A61F 2/95

(52) U.S. Cl. CPC A61F 2/2427; A61F 2/95; A61M 25/0023

(57) ABSTRACT

The present disclosure generally relates to systems, devices, and methods for providing control of fluids from a subject, which may be utilized with an introducer system. Such fluids may comprise blood and may be produced during medical operation upon the subject. An expandable sheath may be provided that is configured to cover a portion of an introducer sheath body between the surface of the subject and a housing of the introducer sheath to retain fluid from the subject within the expandable sheath.

### CROSS-REFERENCE TO RELATED APPLICATIONS

This application is a continuation of PCT patent application no. PCT/US2021/013092, filed on Jan. 12, 2021, which application claims priority to and the benefit of U.S. Provisional Application No. 62/961,845, filed Jan. 16, 2020, each of these applications being incorporated herein by reference in its entirety.

### TECHNICAL FIELD

The present disclosure describes systems, devices, and methods related to implant deployment in fluidic systems and fluid control mechanisms.

### BACKGROUND

A variety of maladies may affect an individual's body. Such maladies may be of the individual's heart, and may include maladies of the individual's heart valves, including the aortic, mitral, tricuspid, and pulmonary valves. Stenosis, for example, is a common and serious valve disease that may affect the operation of the heart valves and an individual's overall well-being.

Implants may be provided that may replace or repair portions of a heart. Prosthetic implants, such as prosthetic heart valves, may be provided to replace a portion of a heart. Prosthetic aortic, mitral, tricuspid, and even pulmonary valves may be provided.

Implants may be deployed to the desired portion of the subject percutaneously, in a minimally invasive manner. Such deployment may occur transcatheter, in which a catheter may be deployed through the vasculature of an individual.

During entry of delivery apparatuses into a subject, an introducer system may be utilized. Such introducer systems may include an introducer sheath and an introducer body configured to be passed through an insertion point in the individual's body. The introducer sheath may form an initial pathway for the delivery apparatus into the subject. However, errors in insertion or issues surrounding removal of the introducer sheath may cause fluid, particularly blood, to leak from the entry point. Improvements in introducer systems are accordingly desired.

### SUMMARY

The present systems and methods relate to systems and methods for providing fluid control during various procedures, including (but not limited to) medical and training procedures. Fluid control includes (but is not limited to) reducing fluid leak, particularly from an introducer system and more particularly from an introducer sheath. In embodiments herein, a system is provided including an introducer sheath configured to be inserted through a surface of a subject. The introducer sheath may include a sheath body having a distal end and a proximal end, and a housing positioned at the proximal end of the sheath body. The system may include an expandable sheath configured to cover a portion of the sheath body between the surface of the subject and the housing to retain fluid from the subject within the expandable sheath. Subjects include (but are not limited to) medical patients, veterinary patients, animal models, cadavers, and simulators of the cardiac and vasculature system (e.g., anthropomorphic phantoms and explant tissue).

In embodiments herein, a system is provided including an introducer sheath including a sheath body having a distal end and a proximal end and configured to be inserted through an insertion point on a surface of a subject. The system may include a wiper body configured to be positioned on the sheath body between the insertion point and the proximal end of the sheath body and slide against the sheath body in a relatively distal direction to reduce fluid from transferring proximal of the wiper body.

In embodiments herein, a method is provided including withdrawing a sheath body of an introducer sheath from an insertion point on a surface of a subject, the sheath body having a distal end and a proximal end. The method may include expanding an expandable sheath along a portion of the sheath body between the insertion point and the proximal end of the sheath body with the sheath body being positioned within an interior cavity of the expandable sheath for retaining fluid from the subject.

In embodiments herein, a method is provided including withdrawing a sheath body of an introducer sheath from an insertion point on a surface of a subject. The method may include sliding a wiper body against the sheath body in a distal direction relative to the sheath body while the sheath body is withdrawn from the insertion point on the surface of the subject.

### BRIEF DESCRIPTION OF THE DRAWINGS

These and other features, aspects, and advantages are described below with reference to the drawings, which are intended to illustrate, but not to limit, the disclosure. In the drawings, like reference characters denote corresponding features consistently throughout similar embodiments.

FIG. 1 is a side view of an introducer system.

FIG. 2 is a side view of an introducer sheath.

FIG. 3 is a cross sectional view of the introducer sheath along line A-A in FIG. 2.

FIG. 4 is a cross sectional view of the introducer sheath along line B-B in FIG. 2.

FIG. 5 is a perspective view of the introducer sheath inserted through a subject's skin.

FIG. 6 is a perspective view of the introducer sheath shown in FIG. 5 being partially withdrawn.

FIG. 7 is a side view of an expandable sheath according to an embodiment of the present disclosure.

FIG. 8 is a perspective view of the expandable sheath shown in FIG. 7.

FIG. 9 is a cross sectional view of the expandable sheath shown in FIG. 7 along line C-C.

FIG. 10 is a cross sectional view of the expandable sheath shown in FIG. 7 along line D-D.

FIG. 11 is a perspective view of the expandable sheath shown in FIG. 7 compressed and extending around a sheath body of the introducer sheath.

FIG. 12 is a perspective view of the expandable sheath shown in FIG. 7 expanded from the position shown in FIG. 11.

FIG. 13 is a front perspective view of a wiper body according to an embodiment of the present disclosure.

FIG. 14 is a rear perspective view of the wiper body shown in FIG. 13 extending over a sheath body of an introducer sheath according to an embodiment of the present disclosure.

FIG. 15 is a perspective view of a wiper body according to an embodiment of the present disclosure.

FIG. 16 is a perspective view of the wiper body shown in FIG. 15 according to an embodiment of the present disclosure sliding along a sheath body of an introducer sheath.

FIG. 17 is a perspective view of an introducer sheath inserted through a subject's skin, with a wiper body shown in cross section.

FIG. 18 is a perspective view of an introducer sheath inserted through a subject's skin, with a wiper body shown in cross section.

### DETAILED DESCRIPTION

The following description and examples illustrate some example embodiments of the disclosure in detail. Those of skill in the art will recognize that there are numerous variations and modifications of the disclosure that are encompassed by its scope. Accordingly, the description of a certain example embodiment should not be deemed to limit the scope of the present disclosure.

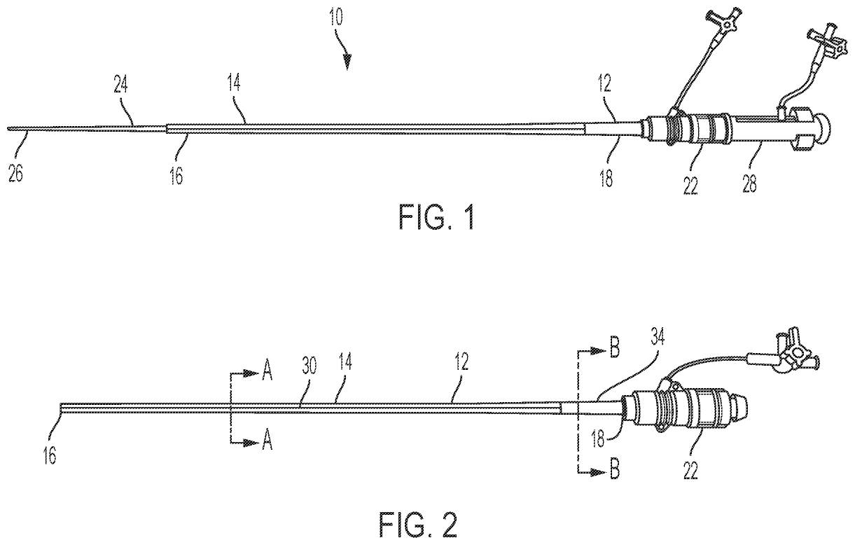

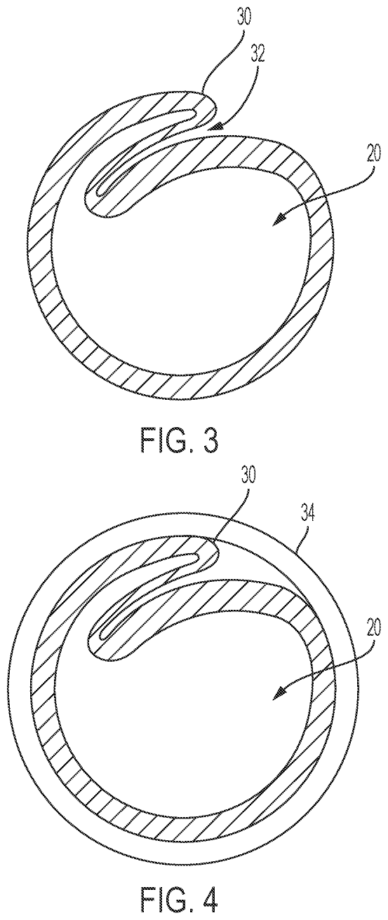

FIG. 1 illustrates an embodiment of an introducer system 10 that may be utilized according to embodiments disclosed herein. The introducer system 10 may include an introducer sheath 12 that may include a sheath body 14 having a distal end 16 and a proximal end 18. The introducer sheath 12 may be configured to be inserted through a surface of a subject at an insertion point. The sheath body 14 may have a length between the distal end 16 and the proximal end 18. The sheath body 14 may include a central interior lumen or cavity 20 (marked in FIG. 3) that a delivery apparatus may be passed through to move to a location within a subject. For instance, the delivery apparatus may be configured to deploy an implant to a heart, such as a heart valve. The implant may comprise a prosthetic heart valve or another form of implant. Other devices may be delivered by the delivery apparatus as desired. In certain embodiments, the delivery apparatus may comprise another form of device for insertion into the subject. The distal end 16 of the sheath body 14 may define an opening for the delivery apparatus to pass distally through.

A housing 22 may be positioned at the proximal end 18 of the sheath body 14. The housing 22 may be configured for an individual to grip during use of the introducer sheath 12. The housing 22 may include ports and valves or the like for controlling fluid flow through the introducer sheath 12 and the housing 22. The housing 22 may be configured to remain exterior of the subject as the introducer sheath 12 is inserted into the subject.

The introducer sheath may be configured to receive an introducer body 24 shown in FIG. 1 in position within the introducer sheath 12. The introducer body 24 may have a distal end 26 that may protrude from the distal end 16 of the introducer sheath 12. A proximal end of the introducer body 24 may include a housing 28. The housing 28 may comprise a grip for control of the introducer body 24 and may include ports and valves or the like for controlling fluid flow through the housing 28. The introducer body 24 may be configured to have a tapered profile that tapers downward from the proximal end of the introducer body 24 to the distal end 26 of the introducer body. The introducer body 24 may be utilized to improve access into vasculature of the subject via the tapered profile. Thus, the introducer body 24 may be introduced into the individual's body with the distal end 26 leading, and the gradual taper of the introducer body 24 may ease access to the vasculature.

The introducer body 24 and introducer sheath 12 may together be inserted into the subject (possibly over a guide wire or the like) and then the introducer body 24 may be withdrawn proximally to leave the introducer sheath 12 in position within the vasculature. The introducer sheath 12 may remain present to guide access of a delivery apparatus, or another device, into the vasculature.

The vasculature may comprise the blood vessels of the subject which may include the femoral artery or other vessels of the subject. The vasculature, such as the femoral artery, may be narrow or stiff, and may be difficult to easily insert a delivery apparatus therein. For example, the delivery apparatus may be larger than the vasculature, or may be unwieldy to penetrate through the skin or vasculature to pass therethrough by itself. Also, the vasculature may be too fragile to receive the delivery apparatus without use of an introducer sheath.

As the initial entry into the subject is preferably minimally invasive and atraumatic as possible, the diameter of the introducer sheath 12 may be relatively narrow upon entry into the vasculature. However, the diameter required to pass a delivery apparatus or the like through the introducer sheath 12 may be larger than this initial diameter. As such, and referring to FIG. 2, the introducer sheath 12 may include a seam 30 along the sheath body 14 that allows the diameter of the sheath body 14 to expand.

FIG. 3, for example, illustrates a cross sectional view of the sheath body 14 along line A-A in FIG. 2. A seam 30 on the outer surface of the sheath body 14 is visible, as a fold of material that forms a channel 32 on an exterior of the sheath body 14. The seam 30 is present to allow the sheath body 14 to expand radially outward to accommodate a diameter of the delivery apparatus or other device. As such, the fold of material may expand outward to increase the effective size of the central lumen or cavity 20.

The seam 30 may extend for the entire length of the sheath body 14, from the distal end 16 to the proximal end 18 marked in FIG. 2. A portion of the sheath body 14, however, may be covered with a proximal sheath 34, which may be in the form of a strain relief. In certain embodiments, the seam 30 may extend for at least a portion of the length of the sheath body 14.

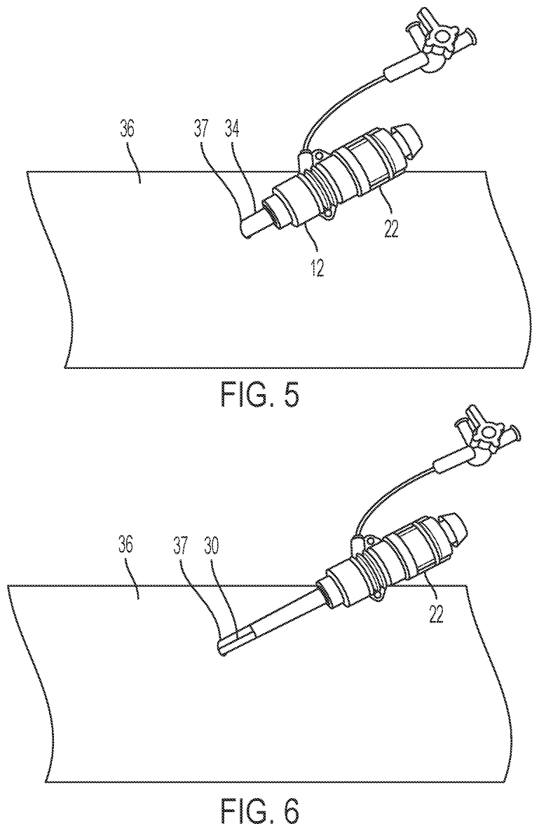

FIG. 4, for example, illustrates a cross sectional view along line B-B in FIG. 2 of the sheath 34 extending over the proximal portion of the seam 30. The sheath 34 may prevent fluid (such as blood) from extending from the seam 30 and out of the subject when the introducer sheath 12 is positioned therein. FIG. 5, for example, illustrates the introducer sheath 12 inserted through the skin 36 into the subject. The insertion point 37 may be relatively small, such that fluid (such as blood) does not leak outward from the subject in large amounts (some leak may be possible). However, if the introducer sheath 12 is inserted incorrectly, or the introducer sheath 12 is inadvertently withdrawn, or is otherwise withdrawn, then the seam 30 may be exposed outside of the subject as shown in FIG. 6.

FIG. 6 illustrates the seam 30 exposed exterior of the subject. The channel 32 marked in FIG. 3 may allow fluid to leak outward, which may result in blood or other fluid loss for the subject or provide a messy insertion point that may make it difficult to perform the desired operation. Further, generally as the introducer sheath 12 is withdrawn, the seam 30 may retain blood or other fluid that may provide a messy device withdrawn from the subject.

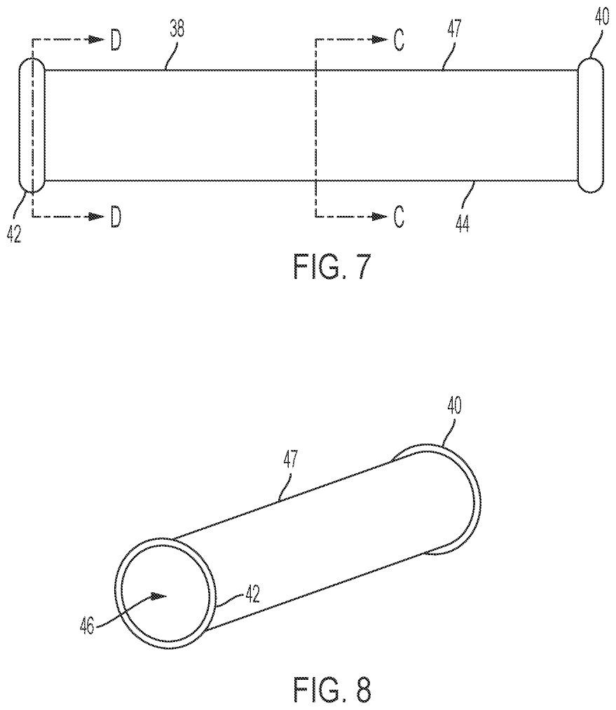

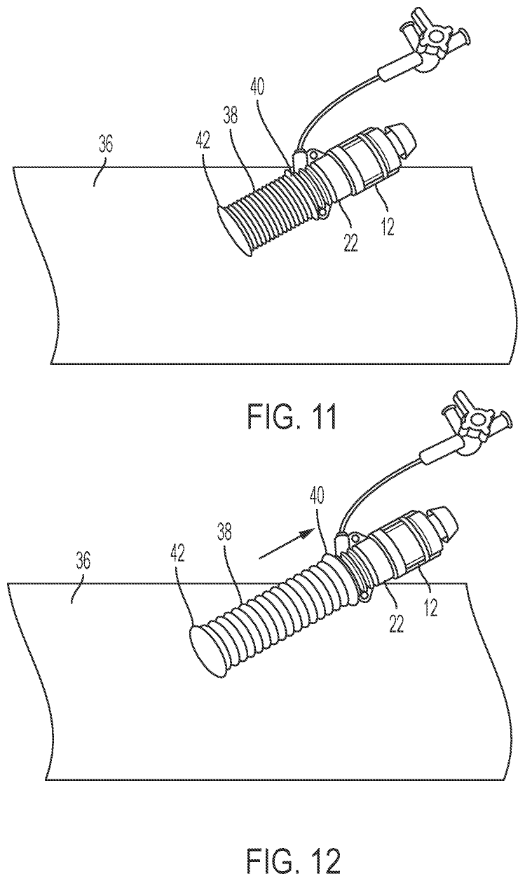

FIG. 7 illustrates an embodiment of an expandable sheath 38. The expandable sheath 38 may be configured to cover a portion of the sheath body 14 between the surface of the subject and the housing 22 of the introducer sheath 12 to retain fluid from the subject within the expandable sheath 38. The expandable sheath 38 may have a proximal portion including a proximal end 40 and a distal portion including a distal end 42 and a length between the ends 40, 42. The expandable sheath 38 is shown in FIG. 7 in an expanded state. However, the sheath 38 may be configured to compress to a compressed state as shown in FIG. 11, in which the expandable sheath 38 has a shorter length between the ends 40, 42. A central body 44 of the expandable sheath 38 may be configured to expand and compress, and accordingly may be made of a flexible material, which may comprise a polymer or other form of flexible material.

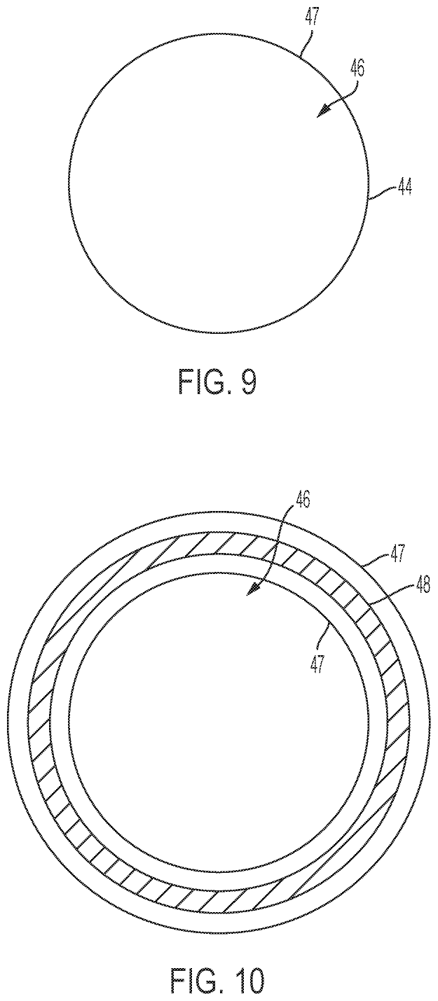

FIG. 9 illustrates a cross sectional view along line C-C in FIG. 7 of the central body 44 showing that the central body 44 surrounds a central interior cavity 46. The central body 44 may include a cylindrical wall 47 that may be relatively thin and may be fluid impermeable. The thin thickness of the central body 44 may allow the central body 44 to expand and compress due to the flexibility provided by the thin thickness.

The interior cavity 46 may be configured to receive the sheath body 14 of the introducer sheath 12 therein and may retain fluid (such as blood) from the subject.

The wall 47 of the expandable sheath 38 may be made of a fluid impermeable material such as a polymer or the like to allow the wall to retain fluid within the central cavity 46.

Referring back to FIG. 7, the distal end 42 of the expandable sheath 38 may include a ring 48 (marked in FIG. 10) that is configured to extend around the insertion point 37 of the introducer sheath 12 into the subject. FIG. 10 illustrate a cross sectional view of the distal end 42 of the expandable sheath. The ring 48 may have the wall of the expandable sheath 38 loop around the ring 48, from the inside of the ring 48 to the outside of the ring 48, to couple to the ring 48 and to maintain a central cavity 46 that does not allow fluid to leak out through the wall 47. The ring 48 may comprise a resilient body such as a body made of an elastic material (e.g., an o-ring) or another form of resilient body. In certain embodiments, a portion of the ring 48 may be uncovered by the wall 47 of the expandable sheath 38 so that the ring 48 may directly contact a portion of the skin to form a seal.

Referring back to FIG. 7, the proximal end 40 of the expandable sheath 38 may include a coupler at the end 40 for coupling to the introducer sheath 12. The coupler may be configured to couple to the housing 22 of the introducer sheath 12. The coupler may comprise a ring (such as ring 48) or another form of coupler for extending over a portion of the introducer sheath 12.

FIG. 8 illustrates a perspective view of the expandable sheath 38.

Referring to FIG. 11, in operation the expandable sheath 38 may be positioned over and surround the sheath body 14 of the introducer sheath 12. The sheath body 14 of the introducer sheath 12 may be positioned within the central cavity 46. The portion including the distal end 42 of the expandable sheath 38 may be pressed towards the surface of the subject, and in many instances, may contact the subject's skin, to form a seal of the central cavity 46 and seal against the surface of the subject. The proximal end 40 of the expandable sheath 38 may extend over a portion of the housing 22 of the introducer sheath 12 to couple to the introducer sheath 12 and may form a seal of the interior of the expandable sheath 38 including the interior cavity 46. The proximal end 40 of the expandable sheath 38 may be coupled to the housing 22.

The expandable sheath 38 may be in a compressed state, compressed axially along a length of the sheath body 14. The expandable sheath 38 may be compressed by the introducer sheath 12 being inserted into the subject as part of the introducer sheath 12 insertion process.

Referring to FIG. 12, the introducer sheath 12 may be withdrawn in a proximal direction and the expandable sheath 38 may expand in length axially along a length of the sheath body 14 as the sheath body 14 is withdrawn from the subject. The expandable sheath 38 may be expanded along a portion of the sheath body 14 between the insertion point 37 and the proximal end 18 of the sheath body 14 with the sheath body 14 being positioned within the interior cavity 46 of the expandable sheath 38 for retaining fluid from the subject. The proximal end 40 may remain coupled to the housing 22 of the introducer sheath 12 and the distal end 42 may remain pressed towards the individual's skin to maintain a seal of the central cavity 46. The introducer sheath 12 may be withdrawn proximally with any fluid (such as blood) from the sheath body 14 and positioned within the seam 30 to be retained within the expandable sheath 38. The expandable sheath 38 is configured to cover at least a portion of the seam 30 as the sheath body 14 is withdrawn from the subject, with the seam 30 positioned within the interior cavity 46. As such, fluid leak from the introducer sheath 12 may be reduced as it is captured by the expandable sheath 38.

The expandable sheath 38 may have a length configured to extend along the entirety of the sheath body 14 from the proximal end 18 to the distal end 16 as the sheath body 14 is withdrawn from the subject. As such, the expandable sheath 38 may be extended to the distal end 16 of the sheath body 14.

The configuration of the expandable sheath and components of the delivery system may be varied in other embodiments.

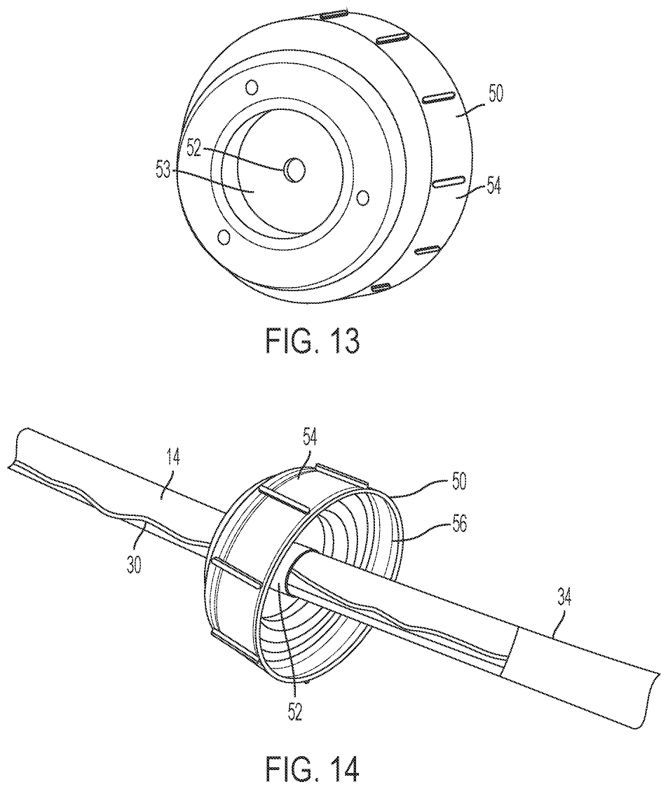

FIG. 13 illustrates a perspective view of a wiper body 50 that may be configured to be positioned on the sheath body 14 between the insertion point 37 and the proximal end 18 of the sheath body 14 and configured to slide against the sheath body 14 in a relatively distal direction to reduce fluid from transferring proximal of the wiper body 50. The wiper body 50 may comprise a ring as shown in FIG. 13 configured to extend around an outer surface of the sheath body 14 and that has an opening 52. The opening 52 may be configured for the sheath body 14 to pass through, with the wiper body 50 pressing against the sheath body 14 to wipe fluid from the sheath body 14. As such, the wiper body 50 may pass along the sheath body 14 to maintain fluid leak towards the distal end of the sheath body 14.

The wiper body 50 may include a housing 54 for an individual to grip and extending around a central portion of the wiper body 50. The central portion of the wiper body may comprise a resilient body 53 configured to press against the outer surface of the sheath body 14. The housing 54 may extend around at least a portion of the resilient body 53.

FIG. 14 illustrates a rear perspective view of the wiper body 50 as the body 50 slides along the sheath body 14. The wiper body 50 may slide along the portion of the sheath body 14 having the seam 30. The wiper body 50 may be pressed distally in a direction towards the skin during withdrawal of the introducer sheath or may be pressed to the skin with the leading surface shown in FIG. 13 in contact with the individual's skin, such that fluid is either pressed back into the subject or maintained locally around the distal side of the wiper body 50 shown in FIG. 13.

The housing 54 may include threading 56 on a proximal side of the wiper body 50 that may be configured to couple to the housing 22. As such, the wiper body 50 may remain coupled to the housing 28 until a time that the wiper body 50 is desired to be used (such as withdrawal of the introducer sheath) to keep the wiper body 50 in position. The wiper body 50 may then be decoupled from the housing 22 and slid distally to maintain fluid distal of the wiper body 50.

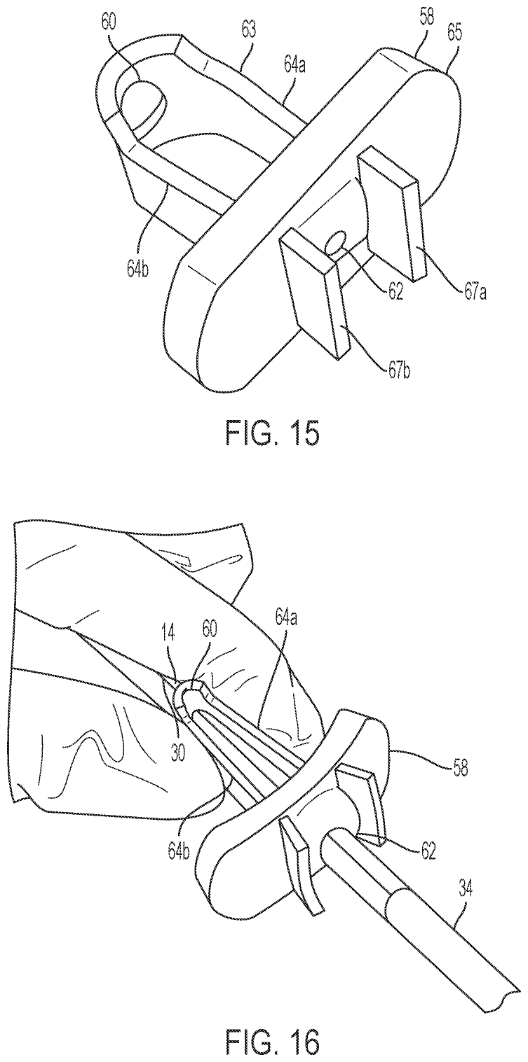

FIG. 15 illustrates an embodiment of a wiper body 58 including two openings 60, 62. The openings 60, 62 may each be configured for the sheath body 14 to pass through. The wiper body 58 may include a grip portion 63 and a connecting body 65. The grip portion 63 may include two arms 64a, 64b that are configured to be gripped by an individual and may include the opening 60. The arms 64a, 64b may extend proximally from the opening 60, longitudinally along opposite sides of the length of the sheath body 14 to end portions 67a, b. The arms 64a, 64b of the wiper body 58 may be configured to slide relatively distally relative to the sheath body to wipe fluid from the wiper body 58 distally.

The connecting body 65 may extend transverse to the direction of extension of the arms 64a, b and may couple the end portions 67a, b of the grip portion 63 together. The connecting body 65 may include the opening 62.

FIG. 16 illustrates the wiper body 58 with the arms 64a, 64b being gripped by an individual and slid relatively distally relative to the sheath body 14. The wiper body 58 may be configured to slide along the sheath body 14, including the portion of the sheath body 14 including the seam 30, to reduce fluid in the seam from transferring proximal of the wiper body 58.

The wiper body 58 may comprise a resilient body configured to press against the outer surface of the sheath body 14.

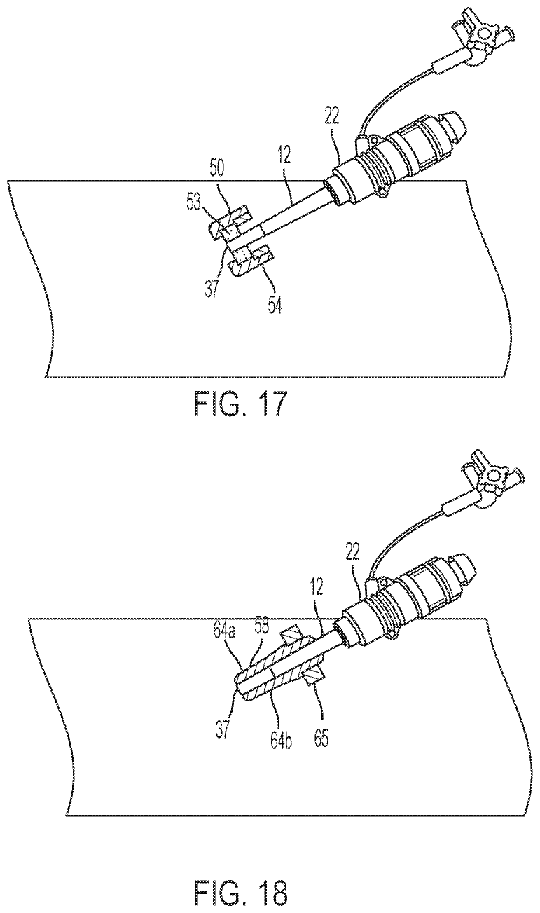

FIGS. 17 and 18 illustrate methods of operation of the wiper body 50 and wiper body 58 respectively. In both FIGS. 17 and 18, the sheath body 14 of the introducer sheath 12 may be withdrawn from an insertion point 37 on a surface of a subject. The respective wiper body 50, 58 may be held in position against the surface of the subject or otherwise be slid against the sheath body 14 in a distal direction relative to the sheath body 14 while the sheath body 14 is withdrawn from the insertion point on the surface of the subject. As such, if the wiper body 50, 58 is held in position against the skin, then as the sheath body 14 is withdrawn, the wiper body 50, 58 will be slid distal relative to the sheath body 14. Further, if the wiper body 50, 58 is moved along the sheath body 14 distally while the sheath body 14 is also being moved proximally, then the wiper body 50, 58 will be slid distal relative to the sheath body 14. The wiper body 50, 58 may be utilized to press fluid from the subject in a distal direction. The wiper body 50, 58 may be pressed against the surface of the subject while withdrawing the sheath body 14 from the insertion point, and may press against the seam 30.

The grip portion, including arms 64a, b may be gripped and the housing 54 may be gripped sliding the respective wiper body 58, 50 against the sheath body. The respective wiper body 58, 50 may be slid to the distal end 16 of the sheath body 14, along the entire length of the sheath body 14.

The configuration of a wiper body and components of the delivery system may be varied in other embodiments.

The use of an expandable sheath and a wiper body is not limited to use with an introducer system or introducer sheath and may extend to use with any medical device to be inserted or withdrawn within a subject. For example, the use may extend to general medical cannula for insertion into a portion of a subject.

An expandable sheath and a wiper body may be utilized in a variety of subjects and procedures. Subjects include (but are not limited to) medical patients, veterinary patients, animal models, cadavers, and simulators of the cardiac and vasculature system (e.g., anthropomorphic phantoms and explant tissue). Procedures include (but are not limited to) medical and training procedures.

The delivery apparatus and the systems disclosed herein may be used in transcatheter aortic valve implantation (TAVI). The delivery apparatus and the systems disclosed herein may be utilized for transarterial access, including transfemoral access, to a heart. In embodiments, various forms of implants may be delivered by a delivery apparatus utilized with system herein, such as stents or filters, or diagnostic devices, among others.

The introducer systems, and introducer sheaths may be utilized in transcatheter percutaneous procedures, including transarterial procedures, which may be transfemoral or transjugular. Transapical procedures, among others, may also be utilized.

Features of embodiments may be modified, substituted, excluded, or combined.

In addition, the methods herein are not limited to the methods specifically described and may include methods of utilizing the systems and apparatuses disclosed herein.

The steps of the method may be modified, excluded, or added to, with systems, apparatuses, and methods disclosed herein.

The features of the embodiments disclosed herein may be implemented independently of the introducer sheaths, or independent of other components disclosed herein. The various apparatuses of the system may be implemented independently.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of systems, apparatuses, and methods as disclosed herein, which is defined solely by the claims. Accordingly, the systems, apparatuses, and methods are not limited to that precisely as shown and described.

Certain embodiments of systems, apparatuses, and methods are described herein, including the best mode known to the inventors for carrying out the same. Of course, variations on these described embodiments will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described embodiments in all possible variations thereof is encompassed by the systems, apparatuses, and methods unless otherwise indicated herein or otherwise clearly contradicted by context.

Groupings of alternative embodiments, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. An introducer sheath system comprising:
an introducer sheath configured to be inserted through a surface of a subject and including:
a sheath body having a distal end and a proximal end, and
a housing positioned at the proximal end of the sheath body; and
an expandable sheath configured to cover a portion of the sheath body between the surface of the subject and the housing to retain fluid from the subject within the expandable sheath.

2. The system of claim 1, wherein the expandable sheath includes a proximal portion and a distal portion, and the distal portion is configured to be pressed towards the surface of the subject.

3. The system of claim 2, further comprising a ring positioned at the distal portion of the expandable sheath and configured to extend around an insertion point of the introducer sheath into the subject.

4. The system of claim 2, further comprising a coupler positioned at the proximal portion of the expandable sheath for coupling with the housing.

5. The system of claim 1, wherein the expandable sheath includes a proximal portion and a distal portion, and the proximal portion includes a seal for sealing an interior of the expandable sheath, and the distal portion includes a seal for sealing against the surface of the subject.

6. The system of claim 1, wherein the expandable sheath is configured to expand axially along a length of the sheath body as the sheath body is withdrawn from the subject.

7. The system of claim 1, wherein the expandable sheath is configured to be compressed axially along a length of the sheath body.

8. The system of claim 1, wherein the sheath body has a length between the distal end and the proximal end of the sheath body, and includes a seam on an outer surface of the sheath body, the seam extending along at least a portion of the length of the sheath body, and the expandable sheath is configured to cover at least a portion of the seam as the sheath body is withdrawn from the subject.

9. The system of claim 8, wherein the expandable sheath is configured to extend to form an interior cavity for retaining fluid from the subject, and the expandable sheath is configured to receive the sheath body within the interior cavity.

10. The system of claim 8, wherein the expandable sheath is configured to extend over an entirety of the sheath body from the proximal end to the distal end.

11. An introducer sheath system comprising:
an introducer sheath including a sheath body having a distal end and a proximal end and configured to be inserted through an insertion point on a surface of a subject; and
a wiper body configured to be positioned on the sheath body between the insertion point and the proximal end of the sheath body and slide against the sheath body in a relatively distal direction to reduce fluid from transferring proximal of the wiper body.

12. The system of claim 11, wherein the wiper body includes an opening configured for the sheath body to be passed through.

13. The system of claim 11, wherein the wiper body comprises a ring configured to extend around an outer surface of the sheath body.

14. The system of claim 11, wherein the wiper body includes a resilient body and a housing configured to be gripped by an individual and extending around at least a portion of the resilient body.

15. The system of claim 11, wherein the wiper body includes a first opening and a second opening, each of the first opening and the second opening being configured for the sheath body to be passed through.

16. The system of claim 11, wherein the sheath body has a length between the distal end and the proximal end of the sheath body, and includes a seam on an outer surface of the sheath body, the seam extending along at least a portion of a length of the sheath body.

17. A method of using an introducer sheath system comprising:
withdrawing a sheath body of an introducer sheath from an insertion point on a surface of a subject, the sheath body having a distal end and a proximal end; and
expanding an expandable sheath along a portion of the sheath body between the insertion point and the proximal end of the sheath body with the sheath body being positioned within an interior cavity of the expandable sheath for retaining fluid from the subject.

18. The method of claim 17, wherein the expandable sheath includes a distal end and a proximal end, and the method further comprises pressing the distal end of the expandable sheath towards the surface of the subject.

19. The method of claim 18, further comprising sealing the distal end of the expandable sheath by pressing the distal end of the expandable sheath towards the surface of the subject.

20. A method of using an introducer sheath system comprising:
withdrawing a sheath body of an introducer sheath from an insertion point on a surface of a subject;
sliding a wiper body against the sheath body in a distal direction relative to the sheath body while the sheath body is withdrawn from the insertion point on the surface of the subject.
